(19) United States
(12) Patent Application Publication
(10) Pub. No.: US 2022/0110838 A1
(43) Pub. Date: Apr. 14, 2022

(54) ORAL CARE NANOPARTICLE COMPOSITIONS AND METHODS

(71) Applicant: EVOQ NANO, INC., Salt Lake City, UT (US)

(72) Inventor: WILLIAM H. NIEDERMEYER, West Jordan, UT (US)

(21) Appl. No.: 17/411,487

(22) Filed: Aug. 25, 2021

Related U.S. Application Data
(60) Provisional application No. 63/071,530, filed on Aug. 28, 2020.

(51) Int. Cl. A61K 8/02; A61K 8/19; A61Q 11/00

(52) U.S. Cl. CPC A61K 8/0245; A61K 8/19; A61K 2800/413; A61K 2800/651; A61Q 11/00

(57) ABSTRACT

A nanoparticle composition for oral care includes at least one of a first set of spherical nanoparticles or a second set of coral shaped nanoparticles, and a stabilizing agent. The nanoparticle composition is added to a carrier suitable for application to an oral cavity, including to teeth and surrounding oral tissues. The nanoparticle composition is configured to control the pH of the microenvironment to which it is applied, thereby preventing and/or treating a variety of oral conditions. The nanoparticle composition can be provided as a concentrated nanoparticle additive addable to a mouthwash, mouth rinse, dentifrice, mouth spray, oral gel, denture cleaning solution, or other carrier suitable for oral application.

### CROSS-REFERENCE TO RELATED APPLICATIONS

This Application claims the benefit of U.S. Provisional Application No. 63/071,530, filed Aug. 28, 2020, which is incorporated by reference in its entirety.

### BACKGROUND

### Field of the Invention

Disclosed herein are nanoparticle compositions and methods for use in oral care, including nanoparticle composition carriers for oral care applications and methods for making and using such compositions.

### 2. Relevant Technology

Dentists and other oral care professionals often recommend oral care compositions to patients to provide the patient with several oral health benefits. For example, oral care compositions can be used to prevent or treat gingivitis and other periodontal diseases, physically remove plaque and tarter (e.g., by brushing and/or use of an abrasive), kill microbes that can cause plaque and tarter buildup, and/or kill microbes that can cause bad breath. Though such compositions are beneficial, periodontal diseases, dental caries, oral malodor, and other undesirable problems associated with oral health persist, and there remains an ongoing need for improved compositions and methods for oral care applications.

Other oral health problems, such as temperature sensitivity, dental abscesses, and canker sores, can also present challenges to oral health. In addition, Herpes labialis (cold sores), caused by a form of herpes simplex virus, is also a common condition affecting the mouth and surrounding tissues. Although there is not yet a cure for herpes infection, outbreaks could be better managed through application of an effective oral care composition, promoting faster healing and pain control.

In addition, suboptimum pH levels within the mouth and/or within microenvironments near teeth or oral tissues can trigger or aggravate a number of undesirable conditions, such as the above mentioned conditions, demineralization of enamel and resultant tooth decay, changes to the microflora within the mouth, increased levels of fermentation products, reduced ability to ward off infectious or otherwise undesirable microbes, increased inflammation, increased healing times of sores, and other undesired effects.

Accordingly, there has been and remains a need to find reliable treatments and prophylactics for use in oral care applications.

### SUMMARY

Disclosed herein are nanoparticle compositions and methods for treating a variety of oral conditions. One or more embodiments include: at least one of a first set of spherical metal nanoparticles having a particle size and a particle size distribution or a second set of coral shaped metal nanoparticles having a particle size and a particle size distribution; and a carrier and/or stabilizing agent; wherein application of the oral care composition to a target area adjusts the pH level at the target area to a target pH level or range.

The spherical and/or coral shaped metal nanoparticles are nonionic and ground state, with no external edges or bond angles. Unlike colloidal silver solutions used in the art for a variety of purposes, the metal nanoparticles do not emit or release ions and are therefore much safer to use and have a different mode of action. It has been found that their nonionic nature improves the ability to penetrate through and break up plaque and/or tartar compared to conventional colloidal silver solutions.

One or more embodiments relate to a concentrated nanoparticle additive addable to a carrier, the additive configured for therapeutically or prophylactically treating or preventing an oral condition, the additive including: at least one of a first set of spherical metal nanoparticles having a particle size and a particle size distribution or a second set of coral shaped metal nanoparticles having a particle size and a particle size distribution; and a stabilizing agent; wherein the additive is configured to be addable to a carrier suitable for application to an oral cavity or surrounding tissue, the first set of spherical metal nanoparticles and/or the second set of coral shaped metal nanoparticles having a concentration to prevent agglomeration of the nanoparticles prior to contact with the carrier, while maintaining effective concentrations after addition to the carrier; and wherein application of the additive and carrier to a target area adjusts the pH level at the target area to a target pH level or range.

One or more embodiments relate to oral care compositions for use in providing a desired oral treatment. Example oral care compositions include dentifrices (e.g., toothpaste, tooth gel), mouth wash, mouth rinse, oral gel, denture cleaning solution, mouth spray, mousse, foam, lozenge, tablet, or dental implement.

One or more embodiments relate to a method of treating an oral condition, the method including: administering a treatment composition to a target area, and the treatment composition controlling a pH imbalance (e.g., a pH level that is too high or too low relative to a target pH level, such as a target pH level of about neutral pH or just below neutral at about 6.5 to 6.9) associated with the oral condition.

These and other advantages and features of the present invention will become more fully apparent from the following description and appended claims, or may be learned by the practice of the invention as set forth hereinafter.

### BRIEF DESCRIPTION OF THE DRAWINGS

FIGS. 1A-1D show TEM images of various non-spherical nanoparticles (i.e., that have surface edges and external bond angles) made according to conventional chemical synthesis or electrical discharge methods;

FIGS. 2A-2C show TEM images of exemplary nonionic spherical-shaped metal nanoparticles (i.e., that have no surface edges or external bond angles), the nanoparticles showing substantially uniform size and narrow particle size distribution, smooth surface morphology, and solid metal cores without the use of coating agents, for use in making nanoparticle compositions for treating oral conditions;

FIGS. 3A-3C show transmission electron microscope (TEM) images of nonionic coral-shaped nanoparticles for use in making nanoparticle compositions for treating oral conditions;

FIGS. 4A and 4B schematically illustrated a proposed mechanism of action by which the nanoparticles can kill or deactivate a microbe, illustrating a microbe protein with disulfide bonds being catalytically denatured by an adjacent spherical-shaped nanoparticle;

FIG. 5 schematically illustrates a mammalian protein with disulfide bonds that are shielded so as to resist being catalytically denatured by an adjacent spherical-shaped nanoparticle;

FIG. 6 is a scanning electron microscope (SEM) image of a human tooth surface treated with water containing no nanoparticles;

FIG. 7 is an SEM image of a human tooth surface treated with a gold coral-shaped nanoparticle and silver spherical-shaped nanoparticle solution;

FIG. 8 is a series of increasingly magnified SEM image of a tooth surface treated with a nanoparticle solution;

FIG. 9 depicts a view of a bacterial cell showing nanoparticles embedded within the bacterium; and

FIG. 10 illustrates the results of conductivity testing comparing various nanoparticle solutions and showing that spherical, metal nanoparticles according to the disclosed embodiments are nonionic.

### DETAILED DESCRIPTION

Disclosed herein are nanoparticle compositions and methods for therapeutically or prophylactically treating and preventing oral conditions, such as periodontal diseases, dental caries, cold sores, canker sores, and other diseases of the mouth, teeth, lips, and other surrounding tissues. Also disclosed herein are nanoparticle compositions and methods for preventing oral conditions or worsening of oral conditions. Also disclosed are methods for making and/or using such nanoparticle compositions.

Oral care compositions of the present disclosure can include one or more metal types of nanoparticles and a carrier. Oral care compositions can be configured for application to the oral cavity (e.g., mouth, teeth, gums, etc.) of a patient or other user for an effective amount of time to treat and/or aid in the prevention of an unwanted oral condition. Oral care compositions are typically not configured to be swallowed or otherwise systemically administered. Examples of oral care compositions include mouthwash, mouth rinse, oral gels, denture cleaning solution, dentifrices (e.g., toothpastes and tooth gels), mouth spray, mousse, foam, lozenge, tablet, dental implement, or other composition capable of being safely applied to the oral cavity.

As used herein, the terms “oral cavity,” “oral tissue,” “oral cavity and surrounding tissues,” “mouth and surrounding tissues,” “treatment area,” “target area,” and the like, refer to teeth, tissues, and other surfaces within the oral cavity or that are near the oral cavity or otherwise associated with the oral cavity, including the teeth, gums, throat, lips, hard and soft palate, tongue, inside surfaces of cheeks, uvula, floor of the mouth, and other tissues and surfaces.

As used herein, the terms “oral condition,” “oral disease,” and the like, refer to diseases and conditions affecting teeth, gums, oral tissues, lips, and/or other surrounding tissues. Examples include periodontal diseases such as gingivitis, dental caries, tooth decay, oral malodor, canker sores, cold sores, cuts, scrapes, abrasions, inflammation, and other undesirable dental or oral conditions.

### Nonionic Metal Nanoparticles

In some embodiments, the metal nanoparticles may comprise or consist essentially of nonionic, ground state metal nanoparticles with no external edges or bond angles. Examples include spherical-shaped metal nanoparticles, coral-shaped metal nanoparticles, or a blend of spherical-shaped metal nanoparticles and coral-shaped metal nanoparticles.

In some embodiments, metal nanoparticles useful for making nanoparticle compositions comprise spherical nanoparticles, preferably spherical-shaped metal nanoparticles having a solid core. The term “spherical-shaped metal nanoparticles” refers to nanoparticles that are made from one or more metals, preferably nonionic, ground state metals, having only internal bond angles and no external edges or bond angles. In this way, the spherical nanoparticles are highly resistant to ionization, highly stable, and highly resistance to agglomeration. Such nanoparticles can exhibit a high -potential, which permits the spherical nanoparticles to remain dispersed within a polar solvent without a surfactant, which is a surprising and unexpected result.

In some embodiments, the spherical-shaped metal nanoparticles are discrete sphere particles having a smooth outer surface. In some instances, the outer surface is completely smooth, generally smooth, and/or somewhat smooth. The nanoparticles are beneficially smooth and thus adhere to other surfaces (i.e., surfaces of the oral cavity) by Van de Waals forces, instead of a covalent chemical bond to hard surfaces. Because of this, the nanomaterials are non-reactive with commonly used dental chemistries. Additionally, because the interaction between the nanoparticles and the target areas is a physical interaction, not a chemical one, any composition comprising the nanoparticles are not hindered when included with existing dental care formulations (e.g., toothpaste, mouth rinse, etc.) or dental care practices (e.g., fluoridation).

In some embodiments, spherical-shaped metal nanoparticles can have a diameter of about 40 nm or less, about 35 nm or less, about 30 nm or less, about 25 nm or less, about 20 nm or less, about 15 nm or less, about 10 nm or less, about 7.5 nm or less, or about 5 nm or less.

In some embodiments, spherical-shaped nanoparticles can have a particle size distribution such that at least 99% of the nanoparticles have a diameter within 30% of the mean diameter of the nanoparticles, or within 20% of the mean diameter, or within 10% of the mean diameter. In some embodiments, spherical-shaped nanoparticles can have a mean particle size and at least 99% of the nanoparticles have a particle size that is within ±3 nm of the mean diameter, ±2 nm of the mean diameter, or ±1 nm of the mean diameter. In some embodiments, spherical-shaped nanoparticles can have ξ-potential of at least 10 mV, preferably at least about 15 mV, more preferably at least about 20 mV, even more preferably at least about 25 mV, and most preferably at least about 30 mV.

Examples of methods and systems for manufacturing spherical-shaped nanoparticles are disclosed in U.S. Pat. No. 9,849,512 to William Niedermeyer, which is incorporated herein by this reference.

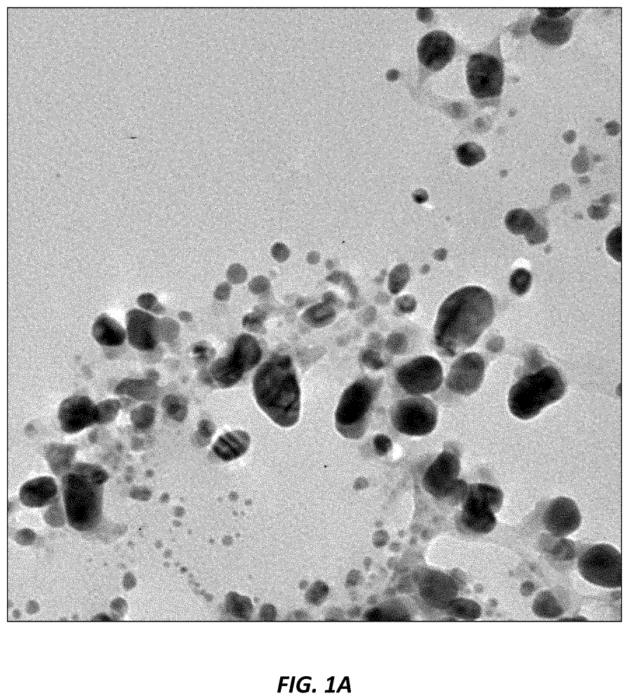

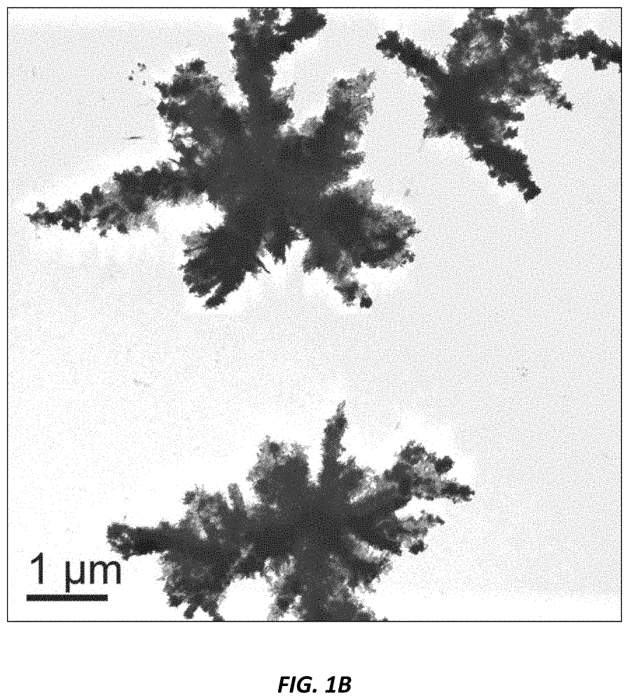

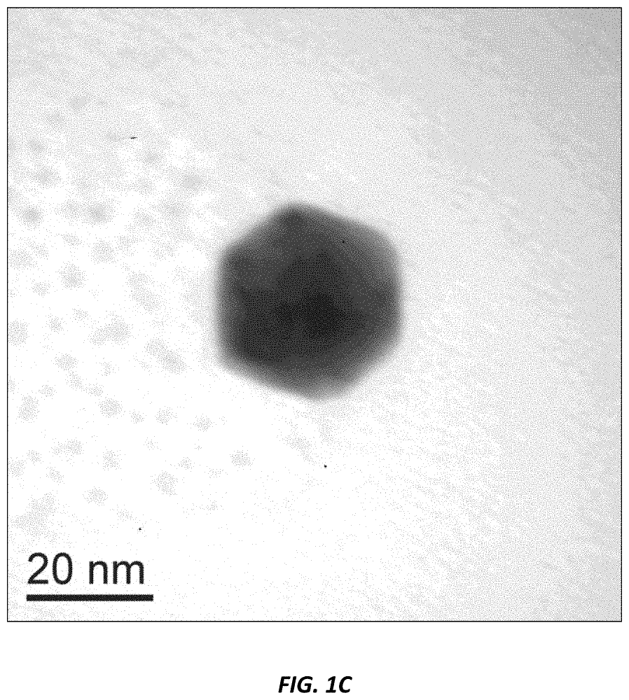

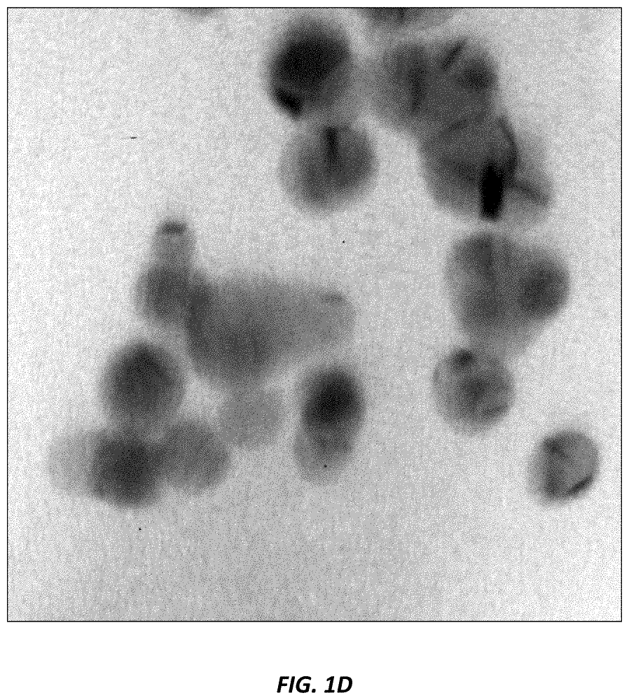

FIGS. 1A-1D show transmission electron microscope (TEM) images of nanoparticles made according to various chemical synthesis methods. As shown, the nanoparticles formed using these various chemical synthesis methods tend to exhibit a clustered, crystalline, faceted, or hedron-like shape rather than a true spherical shape with round and smooth surfaces.

For example, FIG. 1A shows silver nanoparticles formed using a common trisodium citrate method. The nanoparticles are clustered and have a relatively broad size distribution. FIG. 1B shows another set of silver nanoparticles (available from American Biotech Labs, LLC) formed using another chemical synthesis method and showing rough surface morphologies with many edges. Some have called such nanoparticles “nanoflowers”. FIG. 1C shows a gold nanoparticle having a hedron shape as opposed to a truly spherical shape. FIG. 1D shows a set of silver nanoparticles (sold under the trade name MesoSilver), which have relatively smoother surface morphologies but are understood to be shells of silver formed over a non-metallic seed material.

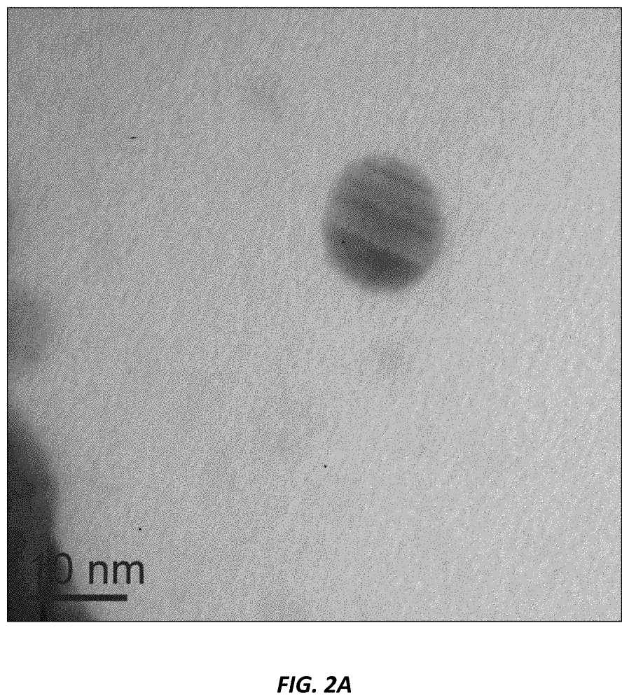

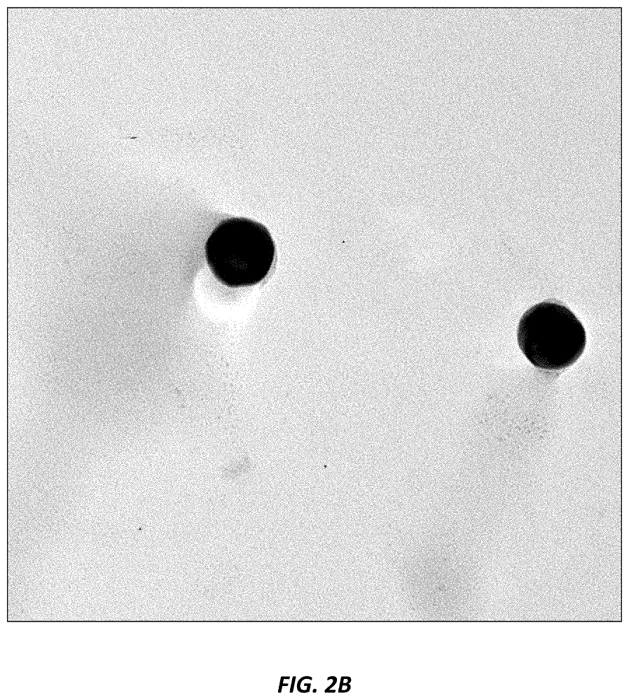

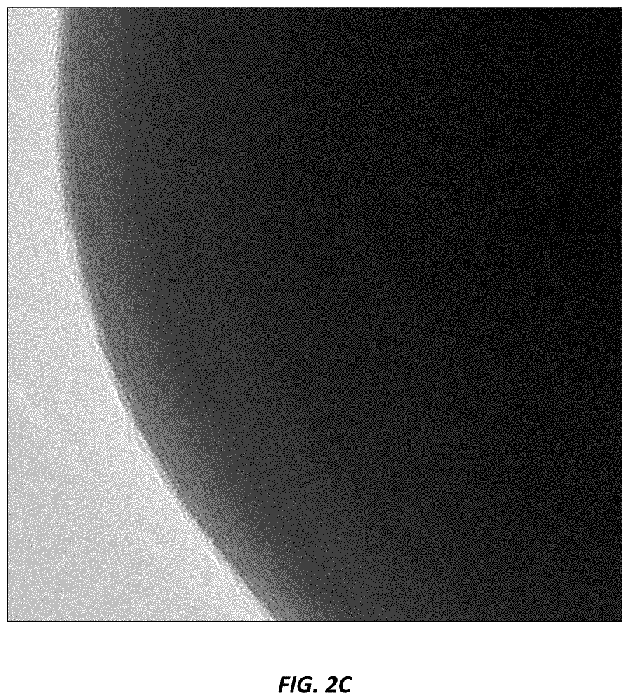

In contrast, the spherical-shaped nanoparticles described herein are solid metal, substantially unclustered, optionally exposed/uncoated, and have a smooth and round surface morphology along with a narrow size distribution. FIGS. 2A-2C show additional TEM images of spherical-shaped nanoparticles. FIG. 2A shows a gold/silver alloy nanoparticle (90% silver and 10% gold by molarity). FIG. 2B shows two spherical nanoparticles side by side to visually illustrate size similarity. FIG. 2C shows a surface of a metal nanoparticle showing the smooth and edgeless surface morphology. The smooth surface assists penetration through plaque and tarter compared to traditional colloidal silver, which is ionic and has external bond angles that promote ionization.

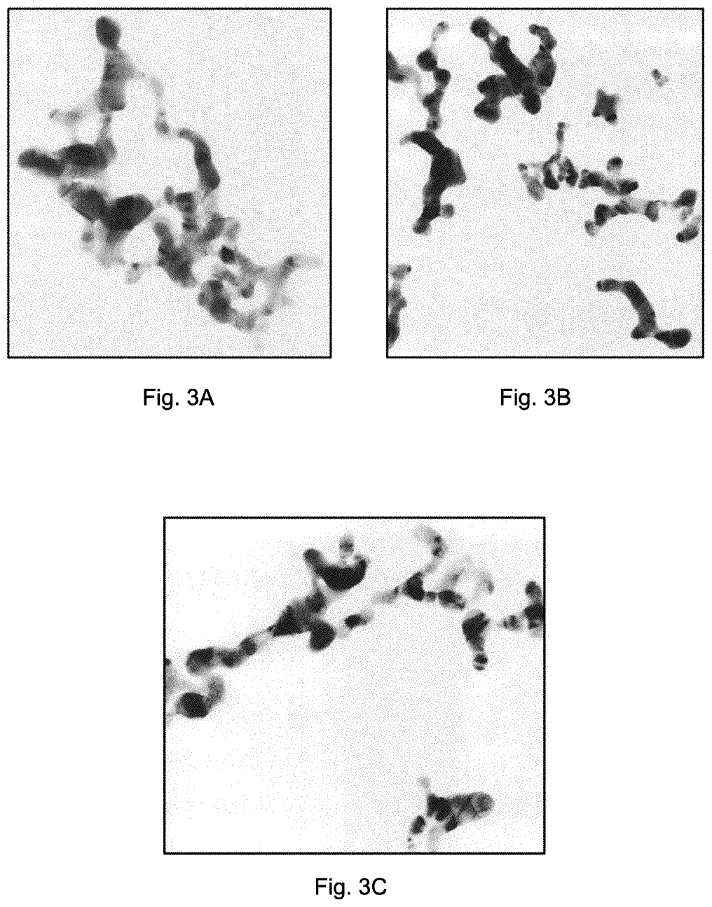

In some embodiments, nonionic metal nanoparticles useful for making nanoparticle compositions may also comprise coral-shaped nanoparticles. The term “coral-shaped metal nanoparticles” refers to nanoparticles that are made from one or more metals, preferably nonionic, ground state metals having a non-uniform cross section and a globular structure formed by multiple, non-linear strands joined together without right angles (see FIGS. 3A-3C). They are not “nanoflowers” and have no physical or chemical resemblance to nanoflowers. Similar to spherical-shaped nanoparticles, coral-shaped nanoparticles may have only internal bond angles and no external edges or bond angles. In this way, coral-shaped nanoparticles can be highly resistant to ionization, highly stable, and highly resistance to agglomeration. Also similar to some embodiments of spherical-shaped nanoparticles, the coral-shaped nanoparticles can be formed as discrete particles having a smooth outer surface, and thus can achieve similar benefits as the spherical-shaped nanoparticles described above. Such coral-shaped nanoparticles can exhibit a high ξ-potential, which permits the coral-shaped nanoparticles to remain dispersed within a polar solvent without a surfactant, which is a surprising and unexpected result.

In some embodiments, coral-shaped nanoparticles can have lengths ranging from about 15 nm to about 100 nm, or about 25 nm to about 95 nm, or about 40 nm to about 90 nm, or about 60 nm to about 85 nm, or about 70 nm to about 80 nm. In some embodiments, coral-shaped nanoparticles can have a particle size distribution such that at least 99% of the nanoparticles have a length within 30% of the mean length, or within 20% of the mean length, or within 10% of the mean length. In some embodiments, coral-shaped nanoparticles can have a ξ-potential of at least 10 mV, preferably at least about 15 mV, more preferably at least about 20 mV, even more preferably at least about 25 mV, and most preferably at least about 30 mV.

Examples of laser-ablation methods and systems for manufacturing coral-shaped nanoparticles are disclosed in U.S. Pat. No. 9,919,363 to William Niedermeyer, which is incorporated herein by this reference.

The metal nanoparticles, including spherical-shaped and coral-shaped nanoparticles, may comprise any desired metal, mixture of metals, or metal alloy, including at least one of silver, gold, platinum, palladium, rhodium, osmium, ruthenium, rhodium, rhenium, molybdenum, copper, iron, nickel, tin, beryllium, cobalt, antimony, chromium, manganese, zirconium, tin, zinc, tungsten, titanium, vanadium, lanthanum, cerium, heterogeneous mixtures thereof, or alloys thereof. Preferred embodiments comprise silver and/or gold nanoparticles.

### Multi-Component Nanoparticle Compositions

In some embodiments, coral-shaped metal nanoparticles can be used in conjunction with spherical-shaped metal nanoparticles. In general, spherical-shaped metal nanoparticles can be smaller than coral-shaped metal nanoparticles and in this way can provide very high surface area for catalyzing desired reactions or providing other desired benefits. On the other hand, the generally larger coral-shaped nanoparticles can exhibit higher surface area per unit mass compared to spherical-shaped nanoparticles because coral-shaped nanoparticles have internal spaces and surfaces rather than a solid core and only an external surface. In some cases, providing nanoparticle compositions containing both spherical-shaped and coral-shaped nanoparticles can provide synergistic results. For example, coral-shaped nanoparticles can help carry and/or potentiate the activity of spherical-shaped nanoparticles in addition to providing their own unique benefits.

In some embodiments, the nanoparticle compositions may include both spherical-shaped and coral-shaped nanoparticles. In some embodiments, the mass ratio of spherical-shaped nanoparticles to coral-shaped nanoparticles in the nanoparticle composition can be in a range of about 1:1 to about 50:1, or about 2.5:1 to about 25:1, or about 5:1 to about 20:1, or about 7.5:1 to about 15:1, or about 9:1 to about 11:1, or about 10:1. The particle number ratio of spherical-shaped nanoparticles to coral-shaped nanoparticles in the nanoparticle composition can be in a range of about 10:1 to about 500:1, or about 25:1 to about 250:1, or about 50:1 to about 200:1, or about 75:1 to about 150:1, or about 90:1 to about 110:1, or about 100:1.

In some embodiments of the present invention, an antimicrobial composition may comprise (1) a first set of metal nanoparticles having a specific particle size and a particle size distribution, (2) and second set of metal nanoparticles having a specific particle size and a particle size distribution, (3) a stabilizing agent, and (4) a carrier, which carrier may be the stabilizing agent itself or may be comprised of one or more other components for delivery of the multicomponent nanoparticles onto and/or into the targeted treatment area within or near the oral cavity of a person or animal.

Some embodiments may include a stabilizing agent. For example, there are times when it is desirable to have different specifically sized nanoparticles within the same solution to take advantage of each of the different properties and effects of the different particles. However, when differently sized particles are mixed into a single solution, the overall long-term stability of these particles within that single solution may be substantially diminished as a result of unequal forces exerted on the various particles causing eventual agglomeration of the particles. This phenomenon may become even more pronounced when that solution is either heated or cooled significantly above or below standard room temperature conditions.

Because of the extremely small size and spherical shape of the nanoparticles it is believed that these particles are quickly absorbed (to be passed out of the body) when applied to the oral cavity of an animal or human. Absorption that is too rapid prevents the nanoparticles from remaining in the oral cavity long enough to provide the desired functions on the teeth and surrounding tissues. Unexpectedly, the inclusion of coral-shaped particles in conjunction with specifically sized spherical nanoparticles has provided increased efficacy for the spherical particles, possibly by preventing the nanoparticles from being absorbed too rapidly and thereby allowing greater exposure of the nanoparticles to the tissues of the oral cavity.

In some embodiments, the compositions will include at least one spherical-shaped nanoparticle component and larger coral-shaped nanoparticle component. In these embodiments, the at least one selected spherical-shaped nanoparticle component will be present in the solution in a range of between about 1 and about 15 ppm (e.g., at least 1 and at most 15 ppm) and more particularly in the range of between bout 1 and about 5 ppm (e.g., at least 1 and at most 5 ppm). Additionally, in some embodiments, the larger coral-shaped nanoparticles will be present in the solution in a range of between about 1 and about 5 ppm (e.g., at least 1 and at most 5 ppm) and more particularly between about 1 and about 3 ppm (e.g., at least 1 and at most 3 ppm). It should be understood that the upper concentration is not restricted as much by efficacy, but more by product formulation cost. Thus, in other embodiments, the spherical-shaped nanoparticle component may present at a concentration above 5 ppm and/or the coral-shaped nanoparticle component may be present at a concentration above 3 ppm.

According to some embodiments, the spherical antimicrobial metal nanoparticles will comprise at least one of silver or gold. In some embodiments, the metal nanoparticles may primarily or exclusively comprise silver. However, in other embodiments, the metal nanoparticles may primarily or exclusively comprise gold. Due to the nature of silver and gold atoms making up the nanoparticles, it has been found that gold nanoparticles are typically better able to hold together at very small sizes (e.g., smaller than about 5-7 nm) compared to silver nanoparticles. On the other hand, a gold-silver alloy typically provides the particle stabilizing activity of gold and the higher activity of silver.

In some embodiments, the coral-shaped nanoparticles will comprise at least one of silver or gold. In some embodiments, the coral-shaped nanoparticles will primarily or exclusively include gold nanoparticles. In some embodiments, the coral-shaped nanoparticles will primarily or exclusively include silver nanoparticles. Alternatively, the coral nanoparticles comprise a gold-silver alloy and/or a mixture of gold and silver nanoparticles.

In some embodiments, nanoparticles comprising silver include a length and/or diameter of about 10 nm with a particle size distribution of about +/−2 nm. In some embodiments, nanoparticles comprising gold include a length and/or diameter of about 25 nm with a particle size distribution of about +/−5 nm. In some embodiments, nanoparticles compositions comprise a mixture of the aforementioned silver and gold nanoparticles.

In some embodiments, the nanoparticles (e.g., spherical and/or coral, silver and/or gold) are formed from a cross ablation process using pure materials without chemicals. In such embodiments, the nanoparticles are formed without any substantial ion emission or production, and the nanoparticles are formed as non-ionic particles.

### Stabilizing Agent/Carrier

The stabilizing agent may itself be beneficial for use in oral care applications. Examples of stabilizing agents include alcohols generally (e.g., ethanol), as alcohols have been observed to effectively maintain nanoparticles of different sizes and different shapes within a given solution. A more particular example of stabilizing agents include polyphenols (e.g., natural-based polyphenols such as arjuna bark extract, grape seed extract, etc.), which can have particular advantages in oral care applications. These natural-based polyphenols typically show good efficacy when dissolved within a carrier in the micro- to milli-molar concentrations range with the upper range limitation typically being constrained not by efficacy but by product cost. In some embodiments, the stabilizing agent is water (e.g., deionized water) or a water and alcohol mixture.

Stabilizing agents can be dissolved into a carrier (e.g., water, alcohol, water alcohol combination, or any combination of other liquid phase materials readily applied to the oral cavity and/or to surrounding tissue of a person or animal).

Additional examples of stabilizing agents include liposomes, creams, and other emulsions. These and similar examples can stabilize the multi-component nanoparticle compositions while constituting the majority of the overall composition, which overall composition may contain little or no water or alcohol or other liquid-phase components. The utilization of gels, creams, and the like that are readily applied to the oral cavity and/or surrounding tissue further facilitates transport of the nanoparticles to one or more target areas where the beneficial effects of those nanoparticles can be achieved.

The various stabilizing agents have the capacity to hold the at least two differently sized and/or shaped nanoparticles in suspension and deliver these nanoparticles to the targeted area of a person or animal (e.g., the oral cavity or surrounding tissues) without so powerfully retaining the nanoparticles so as to diminish the effectiveness of the nanoparticles.

The specific stabilizing agent may be chosen depending on the precise oral condition being treated. For example, in mouthwash or mouth rinse applications, a simple aqueous solution with a stabilizing agent and the appropriate nanoparticles may be preferable over a composition based on a cream formulated with stearic acid, emulsifying wax, boric acid, and/or other ingredients, for example.

Given the ability of many of these stabilizing agents to readily dissolve into water, alcohols and/or oils, introduction or manufacture of the particles into solution with the stabilizing agents allows the multi-component nanoparticles to be readily incorporated into any number of carriers that may then become the basis for a wide array of products including mouthwashes, mouth rinses, oral gels, denture cleaning solutions, dentifrices (e.g., toothpastes and tooth gels), mouth spray, mousse, foam, lozenge, tablet, dental implement, and the like.

Thus, in some embodiments, the nanoparticle composition may also include a carrier, or the stabilizing agent may itself function as a carrier. The carrier can be a liquid, gel, or solid. Some carriers may be more suitable than others depending on the oral condition being treated. For example, the solubility characteristics of the carrier can be selected to maximize or otherwise provide a desired diffusion throughout a treated area within the oral cavity and/or near surrounding tissues. Carriers useful according the present disclosure can include a variety of additional ingredients.

In some embodiments, the composition is in the form of a mouthwash substantially free of ethanol. In some embodiments, the mouthwash has less than about 2% ethanol. In some embodiments, the mouthwash has less than about 1% ethanol. In some embodiments, the mouthwash has less than about 0.5% ethanol. In some embodiments, the composition contains less than about 10% water.

In some embodiments, the compositions further comprise a flavoring and/or sweetener, such as sorbitol.

In some embodiments, the compositions further comprise an effective amount of fluoride. In some embodiments, the fluoride is a salt selected from stannous fluoride, sodium fluoride, potassium fluoride, sodium monofluorophosphate, sodium fluorosilicate, ammonium fluorosilicate, amine fluoride (e.g., N′-octadecyltri-methylenediamine-N,N,N′-tris(2-ethanol)-dihydrofluoride), ammonium fluoride, titanium fluoride, hexafluorosulfate, and combinations of the foregoing.

In some embodiments, the compositions further comprise arginine (e.g., l-arginine) in free or orally acceptable salt form. In some embodiments, the compositions further comprise buffering agents. In some embodiments, the buffering agents are selected from the group sodium phosphate monobasic and disodium phosphate. In some embodiments, the compositions further comprise a humectant. In some embodiments, the humectant is selected from glycerin, sorbitol, propylene glycol, polyethylene glycol, xylitol, and mixtures of the foregoing.

In some embodiments, the compositions further comprise an abrasive or particulate. In some embodiments, the abrasive or particulate is selected from sodium bicarbonate, calcium phosphate, calcium sulfate, precipitated calcium carbonate, silica, iron oxide, aluminum oxide, perlite, plastic particles, and combinations of the foregoing.

Some embodiments provide a dentifrice comprising an abrasive in an amount of about 15 wt. % to about 70 wt. % of the total composition weight.

In some embodiments, the compositions further comprise one or more surfactants. In some embodiments, the surfactants are selected from anionic, cationic, zwitterionic, and nonionic surfactants, and mixtures of the foregoing. In some embodiments, the surfactants are selected from sodium lauryl sulfate, sodium ether lauryl sulfate, and mixtures of the foregoing. In some embodiments, the surfactants are present in an amount from about 0.3% to about 4.5% by weight. In some embodiments, the surfactants are present in an amount from about 0.5% to about 4.0% by weight. In some embodiments, the surfactants are present in an amount from about 1.0% to about 3.5% by weight. In some embodiments, the surfactants are present in an amount from about 1.5% to about 3.0% by weight. In some embodiments, the surfactants are present in an amount from about 2.0% to about 2.5% by weight.

In some embodiments, the compositions further comprise a nonionic surfactant. In some embodiments, the surfactant comprises a poloxamer. In some embodiments, the surfactant is poloxamer 407.

In some embodiments, the compositions further comprise at least one polymer. In some embodiments, the polymer comprises at least one of polyethylene glycols, polyvinylmethyl ether maleic acid copolymers, polysaccharides, polysaccharide gums, or combinations of the foregoing. In some embodiments, the polymer comprises at least one cellulose derivative. In some embodiments, the polymer is selected from carboxymethyl cellulose, xanthan gum, and carrageenan gum, or combinations of the foregoing.

In some embodiments, the compositions comprise one or more antibacterial agents. In some embodiments, the additional one or more antibacterial agents are selected from halogenated diphenyl ethers, herbal extracts, essential oils, bisguanide antiseptics, quaternary ammonium compounds, phenolic antiseptics, hexetidine, octenidine, sanguinarine, povidone iodine, delmopinol, salifluor, metal ions, sanguinarine, propolis, oxygenating agents, phthalic acid and salts and esters, ascorbyl stearate, oleoyl sarcosine, alkyl sulfate, dioctyl sulfosuccinate, salicylanilide, domiphen bromide, delmopinol, octapinol and other piperidino derivatives, nicin preparations, chlorite salts; and combinations and mixtures of any of the foregoing.

In some embodiments, the additional one or more antibacterial agents are selected from triclosan, rosemary extract, tea extract, magnolia extract, thymol, menthol, eucalyptol, geraniol, carvacrol, citral, hinokitol, catechol, methyl salicylate, epigallocatechin gallate, epigallocatechin, gallic acid, miswak extract, sea-buckthorn extract, chlorhexidine, alexidine, octenidine, cetylpyridinium chloride (CPC), benzalkonium chloride, tetradecylpyridinium chloride, N-tetradecyl-4-ethylpyridinium chloride, zinc citrate, hydrogen peroxide, buffered sodium peroxyborate, buffered sodium peroxycarbonate, zinc salts, stannous salts, copper salts, or iron salts, and combinations and mixtures of any of the foregoing.

In some embodiments, the compositions further comprise a whitening agent. In some embodiments, the whitening agent is selected from the group consisting of peroxides, metal chlorites, perborates, percarbonates, peroxyacids, hypochlorites, and combinations of the foregoing.

In some embodiments, the compositions further comprise hydrogen peroxide. In some embodiments, the compositions further comprise a hydrogen peroxide source. In some embodiments, the hydrogen peroxide source is selected from the group consisting of urea peroxide, a peroxide salt, and a peroxide complex. In some embodiments, the hydrogen peroxide source is selected from the group consisting of peroxyphosphate, peroxycarbonate, perborate, peroxysilicate, persulphate salts, calcium peroxyphosphate, sodium perborate, sodium carbonate peroxide, sodium peroxyphosphate, carbamide peroxide, and potassium persulfate.

In some embodiments, the compositions further comprise an agent that interferes with or prevents bacterial attachment. In some embodiments, the agent that interferes with or prevents bacterial attachment is selected from the group consisting of solbrol, chitosan, and combinations of the foregoing.

In some embodiments, the compositions further comprise a source of calcium. In some embodiments, the compositions further comprise a source of phosphate. In some embodiments, the compositions further comprise a source of calcium and phosphate. In some embodiments, the source of calcium and phosphate is selected from the group consisting of calcium-glass complexes, calcium sodium phosphosilicates, calcium-protein complexes, casein phosphopeptide-amorphous calcium phosphate, and combinations of the foregoing.

In some embodiments, the compositions further comprise a soluble calcium salt. In some embodiments, the soluble calcium salt is selected from the group consisting of calcium sulfate, calcium chloride, calcium nitrate, calcium acetate, calcium lactate, and combinations of the foregoing.

In some embodiments, the compositions further comprise a physiologically acceptable potassium salt. In some embodiments, the physiologically acceptable potassium salt is selected from the group consisting of potassium nitrate, potassium chloride, and combinations of the foregoing, in an amount effective to reduce dentinal sensitivity.

In some embodiments, the compositions further comprise a breath freshener (e.g., zinc salts or other breath freshening agents), fragrance, flavoring, or combinations of the foregoing.

The compositions can be formed so as to be applied as a mouth rinse, mouthwash, dentifrice, tooth gel, oral gel, tooth powder, mousse, foam, lozenge, mouth spray, oral tablet, pet care product, and/or dental implement, for example.

In some embodiments, the compositions are effective to (i) inhibit microbial biofilm formation in the oral cavity, (ii) to reduce plaque accumulation, (iii) reduce or inhibit demineralization and promote remineralization of the teeth, (iv) reduce hypersensitivity of the teeth, (v) reduce or inhibit gingivitis, (vi) promote healing of sores or cuts in the mouth, (vii) reduce levels of acid producing bacteria, (viii) to increase relative levels of non-cariogenic and/or non-plaque forming bacteria, (ix) reduce or inhibit formation of dental caries, (x), reduce, repair or inhibit pre-carious lesions of the enamel, e.g., as detected by quantitative light-induced fluorescence (QLF) or electrical caries measurement (ECM), (xi) treat, relieve or reduce dry mouth, (xii) clean the teeth and oral cavity, (xiii) reduce erosion, (xiv) whiten teeth; and/or (xv) promote systemic health, including cardiovascular health, e.g., by reducing potential for systemic infection via the oral tissues.

In one preferred embodiment the carrier may be a cream and would be comprised of a stearic acid cream base optionally containing oils such as coconut or olive oil, grape seed oil or vitamin E oil along with an emulsifying wax which carrier composition acts as the stabilizing agent to maintain the multicomponent nanoparticles within the cream composition. Such a cream can be useful for application to the lips, for example, as in applications for treating and/or preventing cold sores.

In other preferred embodiments the carrier will be a water or combined water and alcohol solution which itself may contain a micro to millimolar concentration of a separate stabilizing agent dissolved into the carrier so as to maintain the multicomponent nanoparticles within the overall composition.

In some embodiments, the metal nanoparticles can be included in a concentration so that a measured quantity of the nanoparticle composition, when applied to a treatment site, will provide a predetermined concentration or quantity of metal nanoparticles and/or will provide ongoing efficacy for an extended period of time. The nanoparticle composition can have a higher concentration of nanoparticles that become diluted when mixed with other liquids applied to or naturally contained on or within the treatment site. Depending on the treatment site, the nature of the nanoparticles being added, and the type of carrier being used, the nanoparticle composition may contain about 0.5 ppm to about 100 ppm of metal nanoparticles by weight, or about 1 ppm to about 50 ppm, or about 2 ppm to about 25 ppm, or about 3 ppm to about 20 ppm metal nanoparticles by weight.

### Concentrated Nanoparticle Additives

In some embodiments, a nanoparticle oral care composition is formulated as a concentrated nanoparticle additive configured so as to be addable to a carrier (e.g., dentifrice, mouthwash, mouth rinse, denture cleaning solution, spray, etc.). For example, a concentrated nanoparticle additive can include (i) between about 10 to about 60 ppm, or about 20 to about 50 ppm, or about 25 to about 40 ppm, or about 30 ppm of a group of spherical metal nanoparticles having a particle size of about 8 nm or less, or about 3 nm to about 14 nm, or about 5 nm to about 13 nm, or about 7 nm to about 12 nm, or about 8 nm to about 10 nm, and (ii) between about 10 to about 120 ppm, or about 25 to about 110 ppm, or about 40 to about 100 ppm, or about 60 to about 90 ppm, or about 80 ppm of a second group of coral metal nanoparticles having a particle size between about 40 and 100 nm.

The concentration of nanoparticles included in the concentrated nanoparticle additive can be configured such that when added to an appropriate carrier, the resulting concentration of nanoparticles remains at an effective level (e.g., about at least 0.5 to 1.5 ppm and/or about at least 1 ppm of spherical nanoparticles and 0.5 ppm of coral-shaped nanoparticles). The concentration of nanoparticles included in the concentrated nanoparticle additive can also be configured such that agglomeration of the nanoparticles is avoided. For example, in some embodiments, a concentrated nanoparticle additive in a deionized water solution can have a concentration as high as about 30 ppm spherical nanoparticles (e.g., silver nanoparticles) and as high as about 80 ppm coral-shaped nanoparticles (e.g., gold nanoparticles) without causing agglomeration.

In some embodiments, concentrations can be even higher without causing agglomeration. For example, in some embodiments, the inclusion of a stabilizing agent (e.g., alcohol) can allow a concentration as high as about 60 ppm spherical nanoparticles (e.g., silver nanoparticles) and as high as about 120 ppm coral-shaped nanoparticles (e.g., gold nanoparticles) without causing agglomeration.

### Regulation of pH

One or more embodiments of the present disclosure are configured to regulate and/or alter the pH of a targeted area within the oral cavity or at tissue near the oral cavity. Such regulation can provide a variety of benefits. For example, dental caries and tooth decay result when the pH level at the teeth drops to acidic levels (e.g., below about 5.5). At lower pH levels, enamel begins to demineralize, and when the pH changes outpace the saliva's ability to buffer, tooth decay will result.

In addition, pH levels can affect the composition of the oral microflora, and when pH levels rise or fall beyond normal levels (e.g., the mouth typically has a pH of slightly below neutral, such as about 6.5 to 6.9), resulting changes in the microflora can lead to a variety of undesirable effects, such as increased levels of fermentation products (e.g., lactic acid), increased production of sulfur compounds resulting in Halitosis, reduced numbers of beneficial microbes and/or reduced microbial diversity within the oral cavity and surrounding tissues (e.g., leading to reduced ability to ward off infectious or undesirable microbes, such as streptococcal bacteria), and/or the triggering of an inflammatory response (e.g., associated with gingivitis). For example, prolonged levels of low pH within the mouth can lead to enhanced colonization of acidogenic bacteria relative to other microbes within the mouth. The greater presence of acidogenic bacteria further decreases the pH, aggravating the undesired effects of substandard pH levels.

Further, pH levels that are too high or too low can negatively affect the healing time of cuts, scrapes, and/or sores within the mouth or in surrounding tissues. For example, pH levels that are higher or lower than typical can lead to increased numbers of harmful microbes in and/or surrounding the wound, increasing the risk of infection and likewise increasing healing times. Changes to a local pH environment may also promote the occurrence of cold sores and/or make it more difficult for the immune system to control the Herpes virus during an outbreak.

In addition, controlling the pH can regulate inflammation within the oral cavity or surrounding tissues. For example, a pH level that is too high or too low can trigger and/or aggravate an inflammatory response, and in some circumstances, this pH-induced triggering and/or aggravating may be independent of any associated microbial infection. Controlling the pH can therefore reduce or eliminate excessive inflammation in the gums or other oral tissues.

Without being bound to a particular theory, the nanoparticle oral care compositions of the present disclosure may be able to interact with the microenvironment to which they are applied (e.g., tooth surfaces, gum surfaces, tissue surfaces within the oral cavity, lips) to control and/or regulate the pH of the microenvironment. For example, the nanoparticle compositions may be able to essentially function as a pH buffer, thereby maintaining pH levels within the microenvironment at or near neutral pH (e.g., about 6.75 to 7.25) or slightly below neutral (e.g., about 6.5 to 6.9). It is theorized that nanoparticle compositions of the present disclosure are able to control and/or regulate the pH of the microenvironment independent of any additional antimicrobial properties of the nanoparticle compositions. That said, the antimicrobial properties of the nanoparticle compositions can further benefit the regulation of pH on the teeth and oral tissues.

For example, the pH control at the surface of the teeth is caused by the ability of the nanoparticles to enter into bacteria, stripping sulfur from the bacteria protoplasm, wherein the sulfur is carried out of the bacterial cell. This process essentially stops the bacteria from processing or replicating without lysing the bacteria. The removal of the sulfur from the bacterial cell nullifies the bacteria. With this nullification, the pH is maintained on the surface of the tooth. This unique mechanism of action is enhanced by the targeted nanoparticle size which reduces the concentrations needed for the desired effect.

### Antimicrobial Activity

Although it is theorized that nanoparticle compositions of the present disclosure are able to control and/or regulate the pH of the microenvironment independent of any additional antimicrobial properties of the nanoparticle compositions, antimicrobial activity can also be a beneficial secondary effect.

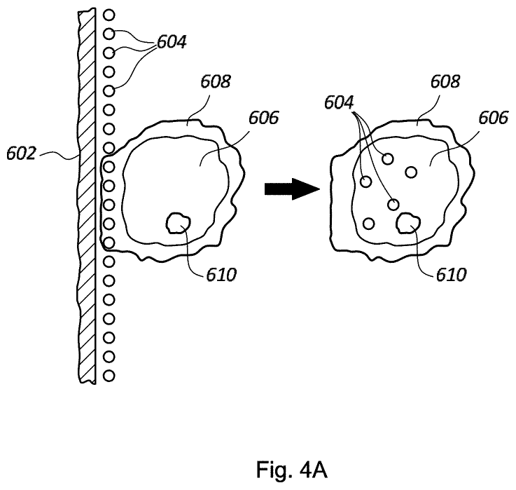

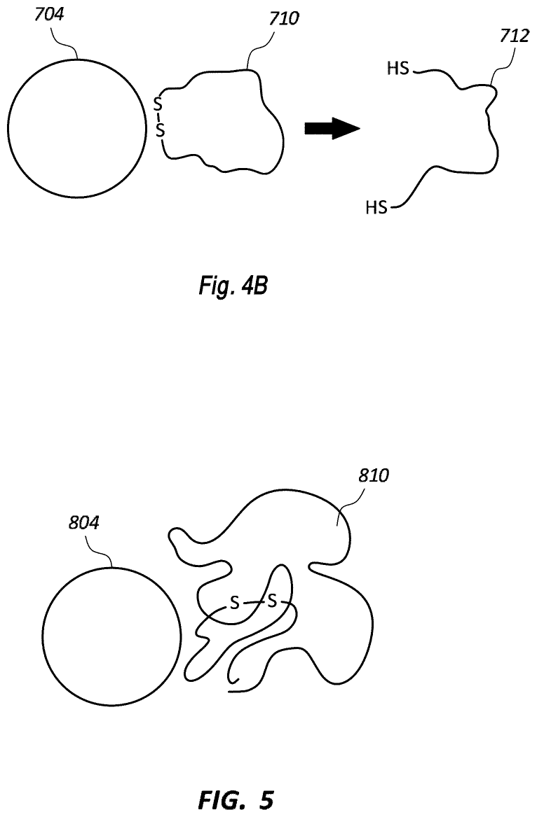

FIG. 4A schematically illustrates a microbe 608 having absorbed spherical-shaped nanoparticles 604 from a solid substrate 602 (e.g., a tooth surface), such as by active absorption or other transport mechanism. Alternatively, spherical-shaped nanoparticles 604 can be provided in a composition (not shown), such as in a liquid or gel carrier. The nanoparticles 604 can freely move throughout the interior 606 of microbe 608 and come into contact with one or more vital proteins or enzymes 610 that, if denatured, will kill or disable the microbe.

One way that nanoparticles may kill or denature a microbe is by catalyzing the cleavage of disulfide (S—S) bonds within a vital protein or enzyme. FIG. 4B schematically illustrates a microbe protein or enzyme 710 with disulfide bonds being catalytically denatured by an adjacent spherical-shaped nanoparticle 704 to yield denatured protein or enzyme 712. In the case of bacteria or fungi, the cleavage of disulfide bonds and/or cleavage of other chemical bonds of vital proteins or enzymes may occur within the cell interior and thereby killing the microbe in this manner. Such catalytic cleavage of disulfide (S—S) bonds is facilitated by the generally simple protein structures of microbes, in which many vital disulfide bonds are exposed and readily cleaved by catalysis.

Another mechanism by which metal (e.g., silver) nanoparticles can kill microbes is through the production of active oxygen species, such as peroxides, which can oxidatively cleave protein bonds, including but not limited to amide bonds.

In the case of viruses, spherical-shaped and coral-shaped metal nanoparticles can alternatively deactivate viruses by attaching to glycoproteins and/or catalyzing protein denaturing reactions in the protein coat so that the virus is no longer able to attach to a host cell and/or inject genetic material into the host cell. Because very small nanoparticles can pass through a virus, denaturing of the protein coat may occur within the interior of the virus. A virus that is rendered unable to attach to a host cell and/or inject genetic material into the host cell is essentially inactive and no longer pathogenic.

Notwithstanding the lethal nature of nonionic metal nanoparticles relative to microbes, they can be relatively harmless to humans, mammals, and healthy mammalian cells, which contain much more complex protein structures compared to simple microbes in which most or all vital disulfide bonds are shielded by other, more stable regions of the protein. FIG. 5 schematically illustrates a mammalian protein 810 with disulfide (S—S) bonds that are shielded so as to resist being catalytically denatured by an adjacent spherical-shaped nanoparticle 804. In many cases the nonionic nanoparticles do not interact with or attach to human or mammalian cells, remain in and follow fluid flow, do not cross barriers, remain in the vascular system, and can be quickly and safely expelled through the urine without damaging kidneys or other cells.

In the particular case of silver (Ag) nanoparticles, the interaction of the silver (Ag) nanoparticle(s) within a microbe has been demonstrated to be particularly lethal without the need to rely on the production of silver ions (Ag⁺) to provide the desired antimicrobial effects, as is typically the case with conventional colloidal silver compositions. The ability of silver (Ag) nanoparticles to provide effective microbial control without any significant release of toxic silver ions (Ag⁺) into the surrounding environment is a substantial advancement in the art. Whatever amount or concentration of silver ions released by silver nanoparticles, if any, is well below known or inherent toxicity levels for animals, such as mammals, birds, reptiles, fish, and amphibians.

As used herein, the modifying term “significant” means that the effect the term is modifying is clinically noticeable and relevant. Thus, the phrase “without significant release of silver ions” means that though there may technically be some small amount of detectable ion release, the amount is so small as to be clinically and functionally negligible. Similarly, the phrase “without significant cell lysis” means that although there may be some observable cell lysis, the amount is negligible and only tangentially related to the actual primary mechanism of cell death/deactivation.

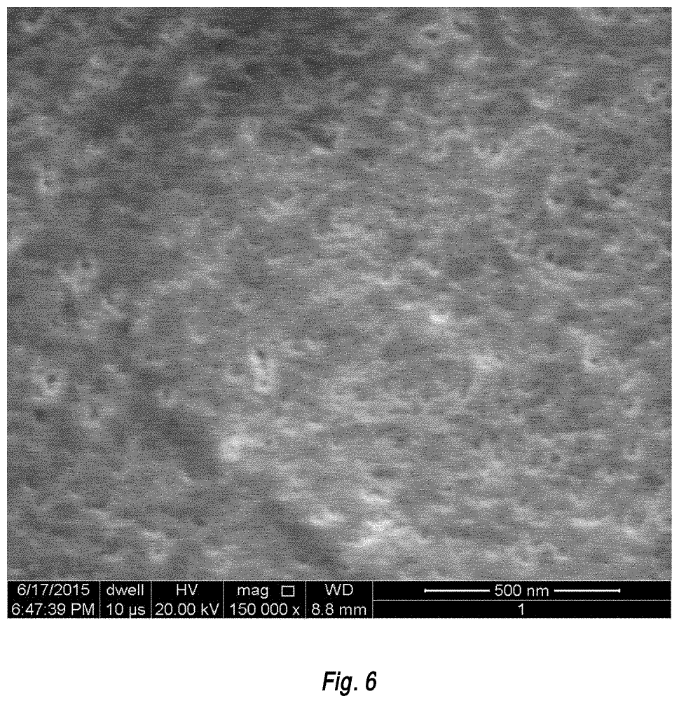

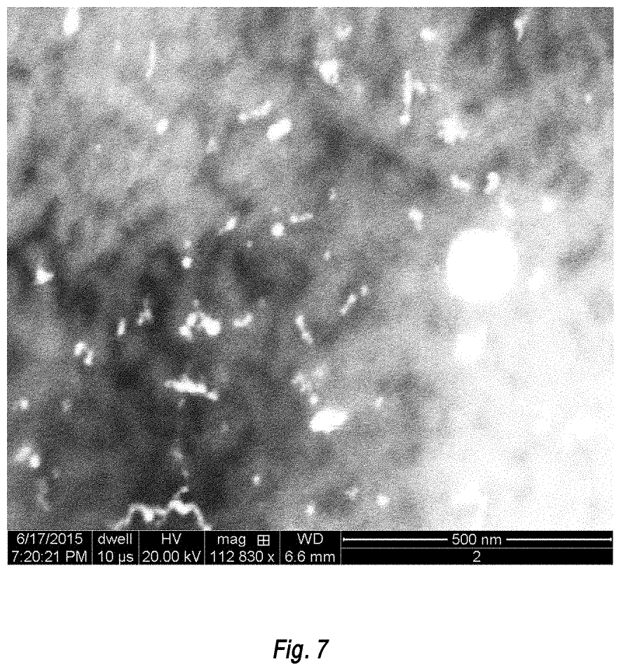

Attention will now be directed to FIGS. 6-9 which show various images of nanoparticles and target areas treated with nanoparticles. For example, FIG. 6 is a scanning electron microscope (SEM) image of a human tooth surface treated with water containing no nanoparticles for comparison. FIG. 7 is an SEM image of a human tooth surface treated with a gold coral-shaped nanoparticle and silver spherical-shaped nanoparticle solution.

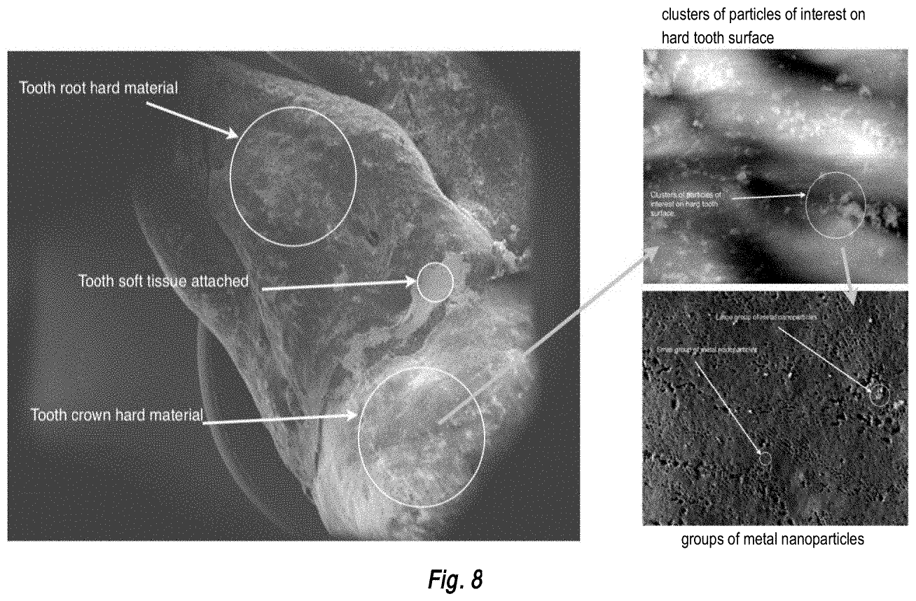

FIG. 8 is a series of increasingly magnified SEM image of a tooth surface treated with a nanoparticle solution. The left image is an oblique view of a portion of tooth showing a tooth root comprising a hard material, areas comprising tooth soft tissue, and a tooth crown comprising a hard material surface. The top right image is magnified view of the tooth crown hard material which shows clusters of particles adhered to the hard tooth surface (e.g., see white clusters of spherical and/or coral shaped nanoparticles). The top bottom image is a further magnified image of the same area of the top right image showing a large group of metal nanoparticles and small group of metal nanoparticles adhered to the tooth surface. In the bottom right image, tooth surface damage can be seen in the dark spots which indicate a porous, surface damage of the tooth. The clusters/groups of nanoparticles adhere to the target areas improving the surface chemistry and physical topography of the tooth.

The nonionic and ground state nature of the metal nanoparticles facilitate penetration through plaque, biofilm, and tarter, as compared to angular and ion-releasing colloidal silver commonly used in the art.

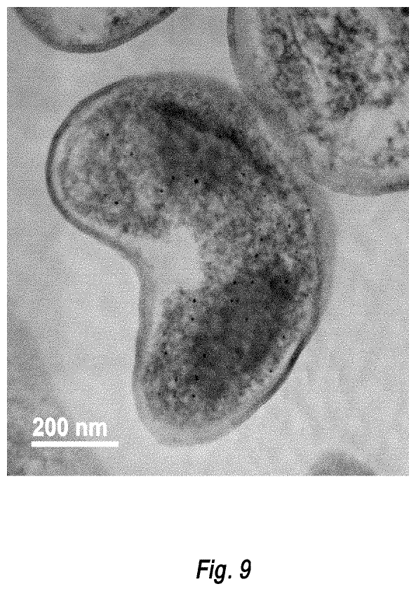

FIG. 9 depicts a bacterium showing nanoparticles embedded within the bacterium. The nanoparticles are shown inside a bacterial cell, wherein the dark spherical spots are the nanoparticles which are believed to catalyze disulfide bond cleavage from inside the cell without lysing the bacterial cell.

### Oral Care Compositions

One or more embodiments relate to oral care compositions for use in providing a desired oral treatment. Example oral care compositions include dentifrices (e.g., toothpaste, tooth gel), mouth wash, mouth rinse, oral gel, denture cleaning solution, mouth spray, mousse, foam, lozenge, tablet, or dental implement.

### Methods of Treatment

In some embodiments, a method of treating an oral condition comprises: (1) applying a treatment composition onto a target area (e.g., one or more teeth or oral tissues) affected by an oral condition, and (2) the treatment composition controlling a pH imbalance associated with the oral condition (e.g., aggravating, underlying, and/or causing the oral condition).

In some embodiments, a method of preventing an oral condition, such as dental caries, comprises: (1) applying a treatment composition onto a target area, and (2) the treatment composition controlling the pH to prevent or reduce the occurrence of the oral condition.

Nanoparticle treatment compositions of the present disclosure may be administered through a variety of different means such using a spray, mouthwash, mouth rinse, dentifrice, denture solution, foam, tablet (e.g., chewable tablet), lozenge, gel, powder, cream, ointment, or other delivery means.

The preferred mode or combination of modes of administration may depend on the type, progression, and/or location of an oral condition. For example, application to teeth to treat and/or prevent dental carries may include application through a dentifrice composition, whereas generalized application to the teeth and/or other oral tissues may include application through a mouthwash or mouth rinse. In another example, application to the lips to treat and/or prevent cold sores and/or gums to treat and/or prevent canker sores may include application through a cream, ointment, wax, gel, and/or balm formulation.

The treatment composition may include spherical-shaped nanoparticles, coral-shaped nanoparticles, or both. In some embodiments, the treatment composition is a multi-component composition including a spherical-shaped nanoparticle component, a coral-shaped nanoparticle component, and a stabilizing agent and/or carrier.

In some embodiments, the treatment is repeated one or more times, or a subsequent, different treatment or combination of treatments is subsequently applied. For example, a treatment may include an increasing or decreasing nanoparticle exposure, such as having a progressively changing nanoparticle concentration with each application to the dermatological condition. The time period between applications may also be established. For example, a nanoparticle composition may be applied weekly, every few days (e.g., five, four, three), every other day, daily, or multiple times per day (e.g., about ten, eight, six, four, or two times per day, or about every hour). In other embodiments, the nanoparticle composition may be applied as needed.

In some embodiments, a method of treating an oral condition includes: (1) administering a treatment composition onto one or more teeth or oral tissues at a treatment area, the treatment composition having (i) between about 1 and about 10 ppm of a group of spherical metal nanoparticles having a particle size of about 8 nm or less, or about 3 nm to about 14 nm, or about 5 nm to about 13 nm, or about 7 nm to about 12 nm, or about 8 nm to about 10 nm, (ii) between about 1 and 10 ppm of a second group of coral metal nanoparticles having a particle size between 40 and 100 nm, and optionally (iii) a milli molar or micro molar concentration of a stabilizing agent, and (2) the oral care composition controlling the pH at the treatment area.

In some embodiments, a method of treating an oral condition includes: (1) adding a concentrated nanoparticle additive to a carrier, the concentrated nanoparticle additive having (i) between about 10 to about 60 ppm, or about 20 to about 50 ppm, or about 25 to about 40 ppm, or about 30 ppm of a group of spherical metal nanoparticles having a particle size of about 8 nm or less, or about 3 nm to about 14 nm, or about 5 nm to about 13 nm, or about 7 nm to about 12 nm, or about 8 nm to about 10 nm, and (ii) between about 10 to about 120 ppm, or about 25 to about 110 ppm, or about 40 to about 100 ppm, or about 60 to about 90 ppm, or about 80 ppm of a second group of coral metal nanoparticles having a particle size between about 40 and 100 nm, (2) administering the carrier and additive composition onto one or more teeth or oral tissues at a treatment area, and (3) the oral care composition controlling the pH at the treatment area. The concentrations are beneficially kept far below any toxicity level to prevent adverse health effects for users, especially during prolonged or long-term use.

It should be appreciated that the benefits of the disclosed embodiments of nanoparticles and nanoparticle compositions and corresponding methods of treatment include both short-term and long-term benefits. Benefits include stabilizing pH levels on target areas in the oral cavity, nullifying bacterial cells, stabilizing the microflora biome, and facilitating an anti-inflammatory effect. These effects, among others described herein, are experienced upon application of the nanoparticles (or treatment comprising the nanoparticles).

Additionally, because the nanoparticles remain adhered to the target areas for extended periods of time, these effects, among other described herein, are realized over a long-term timeframe. This is especially beneficial when compared to treatments that require consistent and daily practice by the patient/user for full efficacy of the treatment. Oftentimes, patients will not have the discipline to perform daily treatments. Thus, having the benefit of a treatment (e.g., nanoparticle infused dental care product) whose active ingredient (e.g., nanoparticles) remains on the tooth for extended periods of time to achieve long-term treatment benefits is a great advantage of the disclosed embodiments. For example, a patient that would have normally needed to apply a treatment daily for several days or several weeks, may be able to apply a nanoparticle composition once and experience beneficial effects for several days and/or several weeks without reapplication.

### Methods of Manufacture

The preferred embodiment for manufacturing the stabilized multi-component antimicrobial nanoparticle compositions requires manufacturing both nanoparticle components (e.g., in embodiments including two separate nanoparticle components) in liquids that are compatible with the final composition.

For example, in the case of a water, alcohol, or water and alcohol based composition, both the first and second nanoparticle components are manufactured in a water, alcohol, or water and alcohol based solution, and the stabilizing agent is then added to one or both of the nanoparticle components and the nanoparticle components can then be combined to achieve the desired concentrations.

In another example, such as in an embodiment having a cream based composition, the first and second nanoparticle components can be either manufactured into one of the major components of the final composition or made in a water or alcohol (or water alcohol mixture) and diluted into the cream based composition.

For example, stearic acid and oils and emulsifying wax and other minor components may be heated to between 160 and 200° F. in order to create the desired final composition. After this nearly completed cream composition has cooled to under preferably about 105° F., first and second sets of nanoparticles which have preferably been manufactured into a natural-based polyphenol can then be added to complete the final cream composition.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope. Further, elements described in relation to any embodiment depicted and/or described herein may be combinable with elements described in relation to any other embodiment depicted and/or described herein.

The terms “approximately,” “about,” and “substantially” as used herein represent an amount or condition close to the stated amount or condition that still performs a desired function or achieves a desired result. For example, the terms “approximately,” “about,” and “substantially” may refer to an amount or condition that deviates by less than 10%, or by less than 5%, or by less than 1%, or by less than 0.1%, or by less than 0.01% from a stated amount or condition.

In addition, unless expressly described otherwise, all stated amounts (e.g., angle measurements, dimension measurements, etc.) are to be interpreted as being “approximately,” “about,” and/or “substantially” the stated amount, regardless of whether the terms “approximately,” “about,” and/or “substantially” are expressly stated in relation to the stated amount(s).

### EXAMPLES

### Example 1

A nanoparticle oral care composition is prepared and includes a 70% water 30% ethanol solution having (i) 60 ppm spherical Ag nanoparticles with a mean diameter of 10 nm with 99% of these Ag nanoparticles having a diameter within ±1 nm of that mean diameter, and (ii) 120 ppm of coral-shaped Au nanoparticles with a mean length of 80 nm with 99% of these Au nanoparticles having a cross section within ±10 nm of that mean length. This oral care solution is readily applied to a target area for treating and/or preventing an oral condition. Additionally, or alternatively, this oral care solution is used as a concentrated additive to be added to a mouth wash or mouth rinse, dentifrice, denture cleaning solution, or other oral care product.

### Example 2

A nanoparticle oral care composition is prepared and includes a 90% water 10% ethanol solution having (i) 40 ppm spherical Ag nanoparticles with a mean diameter of 10 nm with 99% of these Ag nanoparticles having a diameter within ±1 nm of that mean diameter, and (ii) 100 ppm of coral-shaped Au nanoparticles with a mean length of 40 nm with 99% of these Au nanoparticles having a cross section within ±6 nm of that mean length. This oral care solution is readily applied to a target area for treating and/or preventing an oral condition. Additionally, or alternatively, this oral care solution is used as a concentrated additive to be added to a mouth wash or mouth rinse, dentifrice, denture cleaning solution, or other oral care product.

### Example 3

A nanoparticle oral care composition is prepared and includes a 95% water 5% ethanol solution having (i) 30 ppm spherical Ag nanoparticles with a mean diameter of 8 nm with 99% of these Ag nanoparticles having a diameter within ±1 nm of that mean diameter, and (ii) 80 ppm of coral-shaped Au nanoparticles with a mean length of 25 nm with 99% of these Au nanoparticles having a cross section within ±4 nm of that mean length. This oral care solution is readily applied to a target area for treating and/or preventing an oral condition. Additionally, or alternatively, this oral care solution is used as a concentrated additive to be added to a mouth wash or mouth rinse, dentifrice, denture cleaning solution, or other oral care product.

### Example 4

A nanoparticle oral care composition is prepared and includes a 99% water 1% ethanol solution having (i) 30 ppm spherical Ag nanoparticles with a mean diameter of 10 nm with 99% of these Ag nanoparticles having a diameter within ±1 nm of that mean diameter, and (ii) 80 ppm of coral-shaped Au nanoparticles with a mean length of 40 nm with 99% of these Au nanoparticles having a cross section within ±6 nm of that mean length. This oral care solution is readily applied to a target area for treating and/or preventing an oral condition. Additionally, or alternatively, this oral care solution is used as a concentrated additive to be added to a mouth wash or mouth rinse, dentifrice, denture cleaning solution, or other oral care product.

### Example 5

A nanoparticle oral care composition is prepared in a deionized water solution having (i) 30 ppm spherical Ag nanoparticles with a mean diameter of 10 nm with 99% of these Ag nanoparticles having a diameter within ±1 nm of that mean diameter, and (ii) 80 ppm of coral-shaped Au nanoparticles with a mean length of 60 nm with 99% of these Au nanoparticles having a cross section within ±8 nm of that mean length. This oral care solution is readily applied to a target area for treating and/or preventing an oral condition. Additionally, or alternatively, this oral care solution is used as a concentrated additive to be added to a mouth wash or mouth rinse, dentifrice, denture cleaning solution, or other oral care product.

### Example 6

A nanoparticle oral care composition is prepared in a deionized water solution having (i) 1 ppm spherical Ag nanoparticles with a mean diameter of 10 nm with 99% of these Ag nanoparticles having a diameter within ±1 nm of that mean diameter, and (ii) 0.5 ppm of coral-shaped Au nanoparticles with a mean length of 40 nm with 99% of these Au nanoparticles having a cross section within ±6 nm of that mean length. This oral care solution is readily applied to a target area for treating and/or preventing an oral condition.

### Example 7

A nanoparticle oral care composition is prepared and includes a 90% water 10% ethanol solution having (i) 2 ppm spherical Ag nanoparticles with a mean diameter of 10 nm with 99% of these Ag nanoparticles having a diameter within ±1 nm of that mean diameter, and (ii) 1 ppm of coral-shaped Au nanoparticles with a mean length of 40 nm with 99% of these Au nanoparticles having a cross section within ±1 nm of that mean length. This oral care solution is readily applied to a target area for treating and/or preventing an oral condition.

### Example 8

A nanoparticle oral care composition is prepared and includes a 95% water 5% ethanol solution having (i) 5 ppm spherical Ag nanoparticles with a mean diameter of 15 nm with 99% of these Ag nanoparticles having a diameter within ±1.5 nm of that mean diameter, and (ii) 3 ppm of coral-shaped Au nanoparticles with a mean length of 80 nm with 99% of these Au nanoparticles having a cross section within ±10 nm of that mean length. This oral care solution is readily applied to a target area for treating and/or preventing an oral condition.

### Example 9

A cream based carrier suitable for carrying a multicomponent oral care composition is prepared by heating stearic acid, olive oil, and emulsifying wax to between 160 and 200° F. Nanoparticles are suitably added after cooling the composition to about 105° F.

### Example 10

A nanoparticle oral care composition is prepared by adding to the cream carrier of Example 9 (i) 3 ppm coral shaped Au nanoparticles with a mean length of 80 nm with 99% of these Au nanoparticles having a cross section within ±10 nm of that mean length and (ii) 5 ppm of spherical Ag nanoparticles with a mean diameter of 10 nm with 99% of these Ag nanoparticles having a diameter within ±1 nm of that mean diameter and (iii) 1 millimolar concentration of grape seed oil into which both the Ag and Au nanoparticles are added before the grape seed oil is added to the overall product. The oral care solution is readily applied to the lips to treat and/or prevent cold sores.

### Example 11

As a control, a human tooth was treated with distilled water containing no nanoparticles. An SEM image showing the tooth surface is shown in FIG. 6. The tooth was dried in a vacuum desiccator before SEM imaging.

### Example 12

A human tooth was treated with a gold coral-shaped nanoparticle and silver spherical-shaped nanoparticle solution for 20 minutes then placed in distilled water for 10 days. An SEM image showing the tooth surface is shown in FIG. 7. As shown, nanoparticles are maintained on the tooth surface after 10 days of placement in distilled water, with spherical-shaped silver nanoparticles being visible around and nearby the coral-shaped gold nanoparticles. The tooth was dried in a vacuum desiccator before SEM imaging.

### Example 13

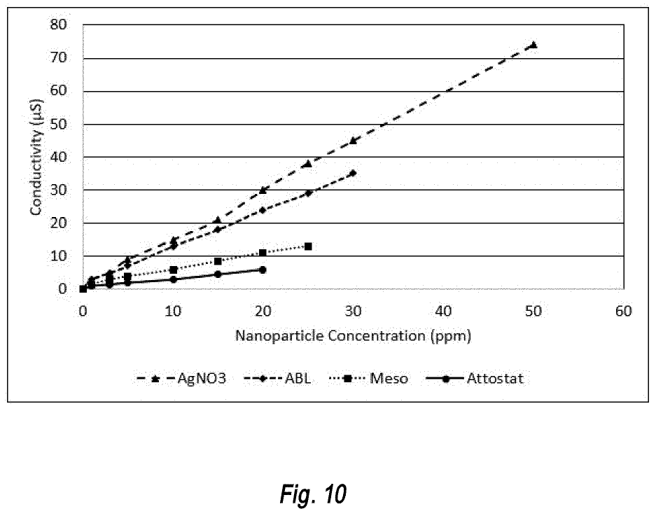

FIG. 10 illustrates the results of conductivity testing comparing various nanoparticle solutions. In Exhibit A, “Attostat” corresponds to spherical-shaped, nonionic silver nanoparticles formed by laser ablation such as described herein, “AgNO₃” is silver nitrate, “Meso” represents a commercially available silver nanoparticle formulation with nanoparticles formed through a chemical reduction process, and “ABL” represents a commercially available silver nanoparticle formulation understood to be formed through an electrolysis process.

The results illustrate that the Attostat nanoparticle formulation had significantly less ion release than any of the other tested nanoparticle formulations. It should be noted that the measured conductivity for Attostat nanoparticle formulations, even at the highest measured concentration of 16 ppm, remained low enough to be on par with typical conductivity measurements for high quality deionized water.

### Example 14

An antibacterial efficacy test was carried out comparing an “Attostat” nanoparticle formulation (8 nm size) against silver nitrate and against the National Institute of Standards and Technology (NIST) Standard Nanocomposix 10 nm silver nanoparticles. The NIST nanoparticles are formed by a chemical reduction process that utilizes citrate as reducing and capping agent. The NIST nanoparticles have a conductivity similar to the “Meso” nanoparticles of Example 13, with detectable but low levels of silver ions.

Relative Light Unit (RLU) counts were recorded at 12 hours and 24 hours post treatment. RLU measurements were carried out using a Hygiena SystemSURE Plus V.2 SN067503 RLU meter with Hygenia AquaSnap TOTAL ATP Water Test Cat#U143 Lot #153019. Culturing media was Hardy Diagnostics Buffered Peptone Water Lot #118272. Samples were prepared with the nanoparticle treatments and then diluted with the media to provide the tested concentrations. The test organism (Microbiologics, E. coli, KwikStik, ATCC#51813, Ref#0791 K, Lot#791-1-6) was incubated in fresh Buffered Peptone Water growth media for 24 hours prior to exposure to the nanoparticle treatments. Tables 1 and 2 illustrate results of RLU counts 12 and 24 hours post nanoparticle treatment, respectively.

TABLE 1
RLU Counts at 12 Hours Post Exposure to Nanoparticle Treatment

| | Attostat | NIST | |
|---|---|---|---|
| | 8 nm | Standard | AgNO3 Silver |
| Concentration | Particles | Particles 10 nm | Nitrate |
| Control 0 ppm (mg/L) | 6256 | 7037 | 6731 |
| 0.25 ppm (mg/L) | 65 | 6908 | 80 |
| 0.5 ppm (mg/L) | 72 | 5416 | 75 |
| 1.0 ppm (mg/L) | 30 | 7189 | 84 |

TABLE 2
RLU Counts at 24 Hours Post Exposure to Nanoparticle Treatment

| | Attostat 8 nm | NIST Standard | AgNO3 Silver |
|---|---|---|---|
| Concentration | Particles | Particles 10 nm | Nitrate |
| Control 0 ppm (mg/L) | 7595 | 5421 | 7342 |
| 0.25 ppm (mg/L) | 25 | 5691 | 25 |
| 0.5 ppm (mg/L) | 8 | 3950 | 46 |
| 1.0 ppm (mg/L) | 30 | 3834 | 30 |

Tables 3 and 4 represent the data in terms of comparing each treatment to its respective control at 12 and 24 hours post treatment, respectively.

TABLE 3
RLU as percentage of control at 12 Hours Post Treatment

| | Attostat 8 nm | NIST Standard | AgNO3 Silver |
|---|---|---|---|
| Concentration | Particles | Particles 10 nm | Nitrate |
| Control 0 ppm (mg/L) | 100% | 100% | 100% |
| 0.25 ppm (mg/L) | 1.0% | 98.2% | 1.2% |
| 0.5 ppm (mg/L) | 1.1% | 77.0% | 1.1% |
| 1.0 ppm (mg/L) | 0.6% | 102.2% | 1.3% |

TABLE 4
RLU as percentage of control at 24 Hours Post Treatment

| | Attostat 8 nm | NIST Standard | AgNO3 Silver |
|---|---|---|---|
| Concentration | Particles | Particles 10 nm | Nitrate |
| Control 0 ppm (mg/L) | 100% | 100% | 100% |
| 0.25 ppm (mg/L) | 0.33% | 105% | 0.34% |
| 0.5 ppm (mg/L) | 0.11% | 72.9% | 0.62% |
| 1.0 ppm (mg/L) | 0.39% | 70.7% | 0.41% |

As shown, at all concentrations tested, the Attostat nanoparticles reduced the number of RLU counts to less than 1.5% from the control baseline at both the 12 hour and 24 hour measurement periods. Anything below 1.5% is below level of accurate detection and is considered a complete kill.

The Attostat nanoparticles effectively reduced RLU counts to below the 1.5% threshold at all tested concentrations. The NIST nanoparticles appeared to show a trend toward greater efficacy at higher concentrations, which would correspond to a normal diffusion model, but even at the highest tested concentration still only reached an RLU count of 70.7% of the initial control baseline at the 24 hour measurement.

The low antimicrobial efficacy of the NIST nanoparticles at the concentrations tested as compared to the silver nitrate could potentially be explained by the lower conductivity, and thus lower ion concentration, of the NIST nanoparticles as compared to the silver nitrate. However, the significant efficacy of the Attostat nanoparticles was surprising given the fact that the Attostat nanoparticles have significantly low to non-detectable levels of ions, even lower than the NIST particles. The Attostat nanoparticles continued to provide antimicrobial activity through the 24 hour testing period with no signs of reduced efficacy.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. An oral care composition for treating or preventing an oral condition, the composition comprising:
spherical-shaped metal nanoparticles having a particle size and a particle size distribution and/or coral-shaped metal nanoparticles having a particle size and a particle size distribution; and
a carrier and/or stabilizing agent;
wherein application of the oral care composition to a target area adjusts the pH level at the target area to a target pH level or range.

2. The oral care composition of claim 1, wherein the stabilizing agent comprises water or an alcohol-water mixture.

3. The oral care composition of claim 1, wherein:
the spherical-shaped metal nanoparticles are provided in a concentration of at least 1 ppm, and/or
the coral-shaped metal nanoparticles are provided in a concentration of at least 0.5 ppm.

4. The oral care composition of claim 1, wherein the target pH level is about neutral pH.

5. The oral care composition of claim 1, wherein the target area is within the mouth, and wherein the target pH level is in a range of about 6.5 to 6.9.

6. The oral care composition of claim 1, wherein the oral care composition is formulated to treat or prevent one or more of dental caries, a periodontal disease, or a sore within the oral cavity.

7. The oral care composition of claim 1, wherein the oral care composition is formulated to treat or prevent a cold sore.

8. The oral care composition of claim 1, the carrier being formed as one or more of a spray, mouthwash, mouth rinse, dentifrice, denture solution, chewable tablet, gel, cream, ointment, balm, or powder.

9. The oral care composition of claim 1, wherein the spherical-shaped nanoparticles are present in the oral care composition in the range of between 1 and 15 ppm or between 1 and 5 ppm.

10. The oral care composition of claim 1, wherein the spherical-shaped nanoparticles have a mean diameter and wherein at least 99% of the spherical-shaped nanoparticles have a diameter within 30% of the mean diameter, or within 20% of the mean diameter, or within 10% of the mean diameter.

11. The oral care composition of claim 1, wherein the spherical-shaped nanoparticles have a mean diameter and wherein at least 99% of the spherical-shaped nanoparticles have a diameter within ±3 nm of the mean diameter, or within ±2 nm of the mean diameter, or within ±1 nm of the mean diameter.

12. The oral care composition of claim 1, wherein the coral-shaped nanoparticles have a particle size of about 40 nm to about 100 nm, each coral-shaped metal nanoparticle having a non-uniform cross section and a globular structure formed by multiple, non-linear strands joined together without right angles, and wherein the coral-shaped nanoparticles have a mean length and wherein at least 99% of the coral-shaped metal nanoparticles have a length within 30% of the mean length, or within 20% of the mean length, or within 10% of the mean length.

13. The oral care composition of claim 1, wherein the coral-shaped nanoparticles are present in the oral care composition in the range of between 1 and 10 ppm or between 1 and 3 ppm.

14. The oral care composition of claim 1, wherein the spherical-shaped nanoparticles metal nanoparticles comprise at least one metal selected from the group consisting of gold, silver, heterogeneous mixtures thereof, and alloys thereof.

15. A concentrated nanoparticle additive for treating or preventing an oral condition, the additive comprising:
spherical-shaped metal nanoparticles having a particle size and a particle size distribution and/or coral-shaped metal nanoparticles having a particle size and a particle size distribution; and
a stabilizing agent,
wherein the additive is configured to be addable to a carrier suitable for application to an oral cavity or surrounding tissue, the spherical-shaped metal nanoparticles and the coral-shaped metal nanoparticles having concentrations that prevent agglomeration of the nanoparticles prior to contact with the carrier while maintaining effective concentrations after addition to the carrier, and
wherein application of the additive and carrier to a target area adjusts the pH level at the target area to a target pH level or range.

16. The additive of claim 15, wherein the spherical-shaped metal nanoparticles have a concentration between about 10 to about 60 ppm and/or the coral-shaped nanoparticles has a concentration between about 10 to about 120 ppm.

17. The additive of claim 15, wherein the stabilizing agent is water, and wherein the spherical-shaped metal nanoparticles have a concentration of 10 ppm or more, or 20 ppm or more, and the coral-shaped metal nanoparticles have a concentration of 10 ppm or more.

18. The additive of claim 15, wherein the stabilizing agent includes an alcohol, and wherein the spherical-shaped metal nanoparticles have a concentration of 10 ppm or more, 30 ppm or more, or 45 ppm or more, and/or the coral-shaped metal nanoparticles have a concentration of 10 ppm or more.

19. A method of treating an oral condition, comprising:
administering a treatment composition as in claim 1 to a target area; and
the treatment composition adjusting a pH imbalance associated with the oral condition to a target pH or range.

20. The method of claim 19, wherein administering the treatment composition is accomplished using one or more of a spray, mouthwash, mouth rinse, dentifrice, denture solution, foam, chewable tablet, lozenge, gel, powder, cream, or ointment.
